(19) United States
(12) Patent Application Publication
(10) Pub. No.: US 2021/0369648 A1
(43) Pub. Date: Dec. 2, 2021

(54) CYSTEAMINE ZINC COMPLEX AND METHOD OF USING A CYSTEAMINE ZINC COMPLEX

(71) Applicant: BIOTHERAVISION LLC, Chicago, IL (US)

(72) Inventors: Roland Bodmeier, Berlin (DE); Chin-Ming CHANG, Zhubei City (TW)

(21) Appl. No.: 17/281,585

(22) PCT Filed: Oct. 4, 2019
(86) PCT No.: PCT/US2019/054873

§ 371 Date: Mar. 31, 2021

Related U.S. Application Data
(60) Provisional application No. 62/742,336, filed on Oct. 6, 2018.

(51) Int. Cl. A61K 31/145; A61K 47/02

(52) U.S. Cl. CPC A61K 31/145; A61K 47/02

(57) ABSTRACT

A method of providing cysteamine to a subject in need of treatment includes administering to the subject an effective amount of a cysteamine-zinc complex, a cysteamine-calcium complex, or a cysteamine-magnesium complex. The disclosure provides a preferred form of cysteamine for oral, topical or parenteral administrations.

### CROSS-REFERENCE TO RELATED APPLICATIONS

This application claims the benefit of U.S. Provisional Application No. 62/742,336, filed Oct. 6, 2018, which is incorporated by reference herein in its entirety.

### FIELD

The present disclosure relates to cysteamine-zinc complexes, cysteamine-calcium complexes, or cysteamine-magnesium complexes having therapeutic properties, together with pharmaceutical compositions comprising the complex, and methods of making and using these complexes and compositions.

### DESCRIPTION OF THE RELATED ART

Drugs containing the thiol moiety play important and diverse roles in biological systems. Thiol drugs contain the —SH functional group, which is the sulfur analog of a hydroxyl or alcohol group. Thiols are also often referred to as mercaptans. Thiols are malodorous substances and highly sensitive to the redox environment and form stable disulfide bonds upon oxidation.

Thiol-containing drugs are incorporated as therapeutic agents in a variety of pharmaceutical preparations and are commonly used as drugs in the treatment of many diseases. Among these compounds, cysteamine, tiopronin, d-penicillamine, captopril, mesna, N-acetylcysteine, thyreostats, thiopurines, alpha-lipoic acid, and their salts and/or prodrugs are the most important.

Cysteamine (synonyms: β-mercaptoethylamine, 2-aminoethanethiol, 2-mercaptoethylamine, decarboxycysteine, thioethanolamine and mercaptamine) is an aminothiol that has been approved as a treatment for cystinosis. It has also been used therapeutically as a radioprotective agent and to prevent severe liver damage after paracetamol poisoning. Its possible use for the treatment for sickle cell anemia, HIV, immunomodulatory agent, systemic lupus erythematosus, and for the treatment of paracetamol hepatotoxicity has also been published.

Cystinosis is a rare genetic autosomal recessive disease due to impaired transport of amino acid cystine across lysosomal membranes. This dysfunction results in continuous intra-lysosomal accumulation of the cystine crystal formation and cellular damage in various tissues. Commonly affected parts of the body include the kidneys (characteristically as Fanconi syndrome in children), eyes, mouth, throat, liver, thyroid, and other organs. Nephropathic cystinosis is associated with kidney that necessitates kidney transplantation. In untreated cystinosis, the inexorable progression of renal glomerular dysfunction leads to uremia and death by 9-10 years of age, unless dialysis or renal transplantation intervenes. Cystinosis is a multi-organ disease that, without sustained medical therapy, will continue to cause significant organ and tissue damage even after kidney transplantation.

As a therapeutic agent for the treatment of cystinosis, cysteamine aims to deplete lysosomal cystine in all body cells and tissues. Cysteamine participates within lysosomes in a thiol-disulfide interchange reaction converting cystine into cysteine and cysteine-cysteamine mixed disulfide, both of which can exit the lysosome in patients with cystinosis.

Cysteamine is the only treatment available for cystinosis and it has been shown to lower intracellular cystine levels. Despite its therapeutic effects, challenges remain to formulate cysteamine into a stable, palatable, and effective dosage form due to its intrinsic poor stability and malodorous properties from the thiol moiety.

As of today, there are two cysteamine oral dosage forms (Cystagon® and Procysbi®) approved for the treatment of nephropathic cystinosis and two ophthalmic eye drops (Cystaran® and Cystadrops®) approved for the treatment of corneal cystine crystal accumulation in patients with cystinosis. Cysteamine acts as a cystine-depleting agent for these treatments.

Cystagon® is an immediate-release oral dosage form containing cysteamine bitartrate. The drug has been approved for clinical use in cystinosis since 1994 in the USA, and in 1997 in Europe. It is the first approved cysteamine oral dosage form. The immediate release formulation of cysteamine is taken at a 6-h interval. Hence, cysteamine is also a potent gastric acid-secretagogue, gastrointestinal complaints such as nausea, dyspepsia, vomiting and epigastric pain are frequent. Cysteamine immediate release dosage form is poorly tolerated. To minimize cysteamine intolerance, the medication is taken at a titration manner started at a low dose and increased gradually to a maintenance dose. The daily maintenance dose is in a range of 400 mg to 2000 mg of cysteamine free base dependent upon patient's body weight or body surface area.

Procysbi® is a twice-daily delayed-release enteric-coated dosage form of cysteamine. It has been approved for clinical use in the USA and Europe since 2013 for the treatment of cystinosis. It was developed based on the observation that direct administration of cysteamine in the small intestine resulted in higher bioavailability in comparison to administration in the stomach or colon. It only needs to be administered twice daily, instead of four times daily. Procysbi® has the potential to improve compliance through its better dosing regimen. However, the poor tolerability of Procysbi® is comparable to that of immediate release dosage form. Moreover, the recommended daily dose for Procysbi® and Cystagon® are the same (1.30 g/m²/day to 1.95 g/m²/day).

While the use of oral cysteamine products is effective to reduce cystine deposition systemically, unfortunately cysteamine from oral administration does not dissolve cystine crystals accumulated in the ocular cornea of cystinosis patients. Topical administration of cysteamine to the cornea has therefore been developed to dissolve cornea cystine crystals. Two cysteamine topical ophthalmic preparations (Cystaran® and Cystadrops®) have been approved for the treatment of corneal cystine crystal accumulation in patients with cystinosis. Both products use cysteamine hydrochloride as the salt form in the preparation.

Cystaran® is the first approved eye-drop preparation. It is composed of a sterile solution of cysteamine hydrochloride 0.65% w/v with benzalkonium chloride and sodium chloride. The product is very unstable and therefore must be kept frozen during long term storage. Once opened, it must be kept under refrigerated condition and must be discarded after one week. Furthermore, due to short duration of action, Cystaran® is recommended to be administered frequently to the eye every awake hour. An ophthalmic viscous solution formulation (Cystadrops®) has recently been developed and proven to be effective when administered 4 times a day. Cystadrops® is a viscous eye drop solution formulation composed of a sterile solution of cysteamine hydrochloride 0.55% w/v with benzalkonium chloride, disodium edetate, carmellose sodium, and citric acid monohydrate. The product is packaged in an amber glass and purged with nitrogen gas bubbling to protect cysteamine from oxidation degradation. While this viscous eye drop solution formulation is relatively more stable in the sealed glass container, its stability quickly diminishes over time once opened, and must be discarded after 7 days. Furthermore, the unconventional package configuration together with cumbersome instructions for opening of the glass vial and attachment of the dropper applicator aseptically adds significant burden and contamination risk to patients.

A lyophilized cysteamine eye drop formulation was developed by Lucane Pharma with an attempt to overcome poor stability, but it failed to obtain regulatory approval for commercialization due to product contamination risk which may have occurred during assembly of the complex container system.

Poor stability of cysteamine in solution is well documented in the literature and storage of it under nitrogen, frozen temperature or freeze dried conditions were recommended. For example,
A. Reda, A. Schepdael, et.al. JIMD Reports. (2017), Pages 47-51.
J. Brouwers. The Lancet. (1976), Page 965.
F. Prior. The Lancet. (1977), 315.
P. Ronald. Journal of Clinical Pharmacy 2 (1978), Pages 199-203.

A number of approaches have been attempted to overcome inherent poor stability and malodorous properties of cysteamine. These are described in, for example, P. Dixon, K. Powell, et.al. (International Journal of Pharmaceutics. 549 (2018), Pages 466-475.S. Pescina, F. Carra, et.al. (European Journal of Pharmaceutics and Biopharmaceutics. 107(2016), Pages 171-179), and others (L. Frost, et.al. European Journal of Medicinal Chemistry. 109 (2016) Pages 206-215; and B. McGaughan, K. Graeme, et.al. Bioorganic Medicinal Chemistry Letters. 18 (2008), Pages 1716-1719.

In summary, current cysteamine preparations with their frequent dosing regimen, poor stability, together with their unpleasant odor and taste, impose a significant burden to cystinosis patients. Thus, there is an unmet medical need and mounting challenges to develop a better cysteamine preparation that improves compliance, tolerability and the quality of life for patients.

Zinc is a common trace element found in many living organisms including humans. It is essential for the normal growth and the reproduction of all higher plants and animals, and of humans. Zinc is found in organs, tissues, bones, fluids, and cells. It is essential for many physiological functions and plays a significant role in a number of enzyme actions in the living systems. Its deficiency may severely affect the homeostasis of a biological system.

Complexes of cysteamine with zinc ions were reported in X. Shu, et.al. Bioinorg Chem Appl. 2016; 2016: 2691253, and Zhou, et.al. Pharmacy and Pharmacology Communications, Volume 6, Issue 7.

Other documents potentially relevant to these problems include: US 20090042850,US 20090175816, and US 20010041193A1.

Use of cysteamine zinc acetate complex as a zinc carrier to accelerate bone healing has also been reported (Gino, et.al., Oral Surg Oral Med Oral Pathol. 1972 September; 34(3):542-52).

Yet the potential use cysteamine zinc complexes to improve stability and other pharmaceutical properties of thiol drugs such as cysteamine in pharmaceutical preparations has never been explored.

### SUMMARY

Some embodiments include a pharmaceutical composition comprising: a thiol drug or a pharmaceutically acceptable salt thereof; zinc or a pharmaceutically acceptable salt thereof, magnesium or a pharmaceutically acceptable salt thereof, calcium or a pharmaceutically acceptable salt thereof, or a combination thereof; and a pharmaceutically acceptable excipient. In some embodiments, these compositions are for treatment of or management of cystinosis disease.

Some embodiments include a method of treating a cystinosis disease, comprising administering a pharmaceutical composition described herein to a mammal, such as a human being, in need thereof.

Some embodiments include a kit comprising the pharmaceutical composition described herein and a label, wherein the label contains instructions to administer the pharmaceutical composition to a mammal or a human being for the treatment of a cystinosis disease.

Some embodiments include use of the pharmaceutical composition described herein in the manufacture of a medicament for the treatment of a cystinosis disease.

Some embodiments include a method of enhancing the stability of a thiol drug in a pharmaceutical composition, comprising combining zinc, or a pharmaceutically acceptable salt thereof, with the thiol drug, or a pharmaceutically acceptable salt thereof, and a pharmaceutically acceptable excipient.

Some embodiments include a method of improving the stability, odor, or taste of a pharmaceutical dosage form containing cysteamine, comprising including in the zinc pharmaceutical dosage form.

Further areas of applicability of the present disclosure will become apparent from the detailed description provided hereinafter. The detailed description and specific examples, while indicating the preferred embodiment of the disclosure, are intended for purposes of illustration only and are not intended to limit the scope of the disclosure.

### BRIEF DESCRIPTION OF THE DRAWINGS

FIGS. 1(a) and 1(b) are graphs showing the stabilization effect of zinc on cysteamine stability. Four preparations were prepared for comparison. Zinc chloride containing preparations were prepared at pH 5.0 and pH 5.5 using hydroxypropyl methylcellulose as viscosity agent. The preparations without presence of zinc chloride were prepared at pH 5.0 with sodium carboxymethylcellulose and at pH 5.5 with hydroxypropyl methylcellulose as viscosity agent, respectively. The graph illustrates assay and impurity level of cysteamine in these preparations at 3-month stability time point stored at 25° C. and 40° C. stability storage conditions.

FIGS. 2(a) and 2(b) are graphs showing the stabilization effect of zinc on cysteamine stability within a pH range of 5 to 6.5. Sodium chloride and hydroxypropyl methylcellulose were used in these preparations as tonicity agent and viscosity agent, respectively. The graph illustrates assay and impurity level of cysteamine at 3-month stability time point stored at 25° C. and 40° C. stability storage conditions.

FIGS. 3(a) and 3(b) are graphs showing the stabilization effect of zinc on cysteamine stability within a pH range of 5.5 to 7. Mannitol and hydroxypropyl methylcellulose were used in these preparations as tonicity agent and viscosity agent, respectively. The graph illustrates assay and impurity level of cysteamine in these preparations comparing to the preparations without presence of zinc chloride at 3-month stability time point stored at 25° C. and 40° C. stability storage conditions.

FIGS. 4(a) and 4(b) are graphs showing the effect of zinc level on cysteamine stability in the preparations formulated at cysteamine:zinc (CH:Zn) molar ratio of 2:1, 4:1 and 8:1. Mannitol and hydroxypropyl methylcellulose were used in these preparations as tonicity agent and viscosity agent, respectively. The graph illustrates assay and impurity level of cysteamine in these preparations at 3-months stability time point stored at 25° C. and over 3 months period at 40° C. stability storage conditions.

### DETAILED DESCRIPTION

One object of the present disclosure is to provide a zinc, magnesium, or calcium containing pharmaceutical dosage form which is capable of improving pharmaceutical dosage properties of drug molecules with a thiol functional group such as cysteamine.

As the results of the extensive studies by the present inventors, it was found that zinc improves the stability of cysteamine and hence solved the decades old challenge to develop cysteamine into a more stable, acceptable and palatable pharmaceutical preparation.

Namely, the present disclosure relates to

(1) a pharmaceutical preparation comprising a thiol drug or its salt and zinc or a salt thereof to enhance physicochemical or organoleptic properties of thiol drug,

(2) the pharmaceutical preparation according to (1), wherein the thiol drug is cysteamine or the salt thereof,

(3) the pharmaceutical preparation according to (1), wherein the zinc salt comprises zinc oxide, zinc bromide zinc chloride, zinc carbonate, zinc gluconate, zinc acetate, zinc phosphate, zinc sulfate or another zinc salt form,

(4) a method of using zinc to enhance the stability of cysteamine or its salt in pharmaceutical preparations,

(5) a method of using zinc in cysteamine pharmaceutical dosage forms where its frequent dosing regimen, poor stability, together with their unpleasant odor and taste, could be addressed to reduce significant burden to cystinosis patients.

Without intending to limit the scope of this disclosure, examples of thiol drugs are cysteamine, tiopronin, d-penicillamine, captopril, mesna, N-acetylcysteine, thyreostats, thiopurines, alpha-lipoic acid, and their salts and/or prodrugs.

Further without intending to limit the scope of this disclosure, the thiol functional group in the drug can be in either oxidized or reduced state.

The present disclosure provides cysteamine in a form of a zinc complex, which is stable, therapeutically effective, and preferred over cysteamine hydrochloride, a bitartrate salt or other salt forms in pharmaceutical preparations.

In one embodiment, the cysteamine zinc complex is included in ophthalmic dosage forms in which the dosage form is more palatable, stable, convenient to use or tolerable than other ophthalmic preparations containing cysteamine. Yet, cysteamine zinc complex is therapeutically effective.

In one embodiment, the cysteamine zinc complex is included in injectable dosage forms in which the dosage form is stable and convenient to use. Yet, cysteamine zinc complex is therapeutically effective.

In one embodiment, the cysteamine zinc complex is included in oral dosage forms in which the dosage form is more palatable, convenient to use or more tolerable than other oral preparations containing cysteamine. Yet, cysteamine zinc complex is therapeutically effective.

In one embodiment, the cysteamine zinc complex is formed in the presence of cysteamine free base or its salt forms with zinc (e.g. zinc oxide, zinc bromide, zinc chloride, zinc carbonate, zinc gluconate, zinc acetate, zinc phosphate, zinc sulfate, etc.) to obtain cysteamine-zinc complexes.

In one embodiment, the cysteamine zinc complex is formed in the presence of cysteamine free base or its salt forms with calcium (e.g. calcium oxide, calcium bromide, calcium chloride, calcium carbonate, calcium gluconate, calcium acetate, calcium phosphate, calcium sulfate, etc.) to obtain cysteamine-calcium complexes.

In one embodiment, the cysteamine magnesium complex is formed in the presence of cysteamine free base or its salt forms with magnesium (e.g. magnesium oxide, magnesium bromide, magnesium chloride, magnesium carbonate, magnesium gluconate, magnesium acetate, magnesium phosphate, magnesium sulfate, etc.) to obtain cysteamine-magnesium complexes.

The following description of the preferred embodiment(s) is merely exemplary in nature and is in no way intended to limit the disclosure, its application, or uses.

The disclosure therefore provides, in a first embodiment, a cysteamine-zinc complex, wherein the complex is formed from a mixture of cysteamine free base or one or more of its salts with zinc or one or more its salts. The molar ratio of cysteamine:zinc may be in a range of about 8:1 to about 1:1, about 1:1-1.5:1, about 1.5:1-2:1, about 2:1-2.5:1, about 2.5:1-3:1, about 3:1-4:1, about 4:1-5:1, about 5:1-6:1, about 6:1-7:1, about 7:1-8:1, about 1:1-2:1, about 2:1-4:1, about 4:1-8:1, about 1.8:1-2.2-1, about 2:1, about 3:1, about 4:1, about 5:1, about 6:1, about 7:1, or about 8:1.

The disclosure therefore provides, in a first embodiment, a cysteamine-zinc complex, a cysteamine-calcium complex, or a cysteamine-magnesium complex, wherein the complex is formed from a mixture of cysteamine free base or one or more of its salts with zinc or one or more its salts. The molar ratio of cysteamine:calcium may be in a range of about 8:1 to about 1:1, about 1:1-1.5:1, about 1.5:1-2:1, about 2:1-2.5:1, about 2.5:1-3:1, about 3:1-4:1, about 4:1-5:1, about 5:1-6:1, about 6:1-7:1, about 7:1-8:1, about 1:1-2:1, about 2:1-4:1, about 4:1-8:1, about 1.8:1-2.2-1, about 2:1, about 3:1, about 4:1, about 5:1, about 6:1, about 7:1, or about 8:1.

The disclosure therefore provides, in a first embodiment, a cysteamine-zinc complex, a cysteamine-calcium complex, or a cysteamine-magnesium complex, wherein the complex is formed from a mixture of cysteamine free base or one or more of its salts with zinc or one or more its salts. The molar ratio of cysteamine:magnesium may be in a range of about 8:1 to about 1:1, about 1:1-1.5:1, about 1.5:1-2:1, about 2:1-2.5:1, about 2.5:1-3:1, about 3:1-4:1, about 4:1-5:1, about 5:1-6:1, about 6:1-7:1, about 7:1-8:1, about 1:1-2:1, about 2:1-4:1, about 4:1-8:1, about 1.8:1-2.2-1, about 2:1, about 3:1, about 4:1, about 5:1, about 6:1, about 7:1, or about 8:1.

These cysteamine-zinc complexes, cysteamine-calcium complexes, or cysteamine-magnesium complexes may have improved stability and may be stable at about 5° C., about 25° C., or at about 40° C., for at least 1 week, at least 2 weeks, at least 3 weeks, at least 1 month, at least 2 months, at least 3 months, at least 4 months, at least 6 months, at least 9 months, at least 12 months, at least 2 years, or more.

In a further embodiment, the disclosure provides a pharmaceutical composition of the cysteamine-zinc complexes, cysteamine-calcium complexes, or cysteamine-magnesium complexes for therapeutic use.

In some embodiments, the pharmaceutical composition of the cysteamine-zinc complexes, cysteamine-calcium complexes, or cysteamine-magnesium complexes may have been stored for at least 1 week, at least 2 weeks, at least 3 weeks, at least 1 month, at least 2 months, at least 3 months, at least 4 months, at least 6 months, at least 9 months, at least 12 months, at least 2 years, or more, at a temperature of about −30° C. to about 40° C., about −30° C. to about 0° C., about 0-20° C., about 20-40° C., about 5° C., or about 25 ® C.

The disclosure further provides the use of a cysteamine-zinc complex, cysteamine-magnesium complex, cysteamine-calcium complex for the treatment of cystinosis.

A cysteamine-zinc complex, cysteamine-magnesium complex, cysteamine-calcium complex can be incorporated into a pharmaceutical dosage form suitable for oral, topical, or parenteral administrations.

A cysteamine-zinc complex, cysteamine-magnesium complex, cysteamine-calcium complex may be formulated into a tablet, capsule, pellets, or drinkable liquid dosage forms for oral administration.

A cysteamine-zinc complex, cysteamine-magnesium complex, cysteamine-calcium complex may be formulated into a solution, emulsion or suspension dosage form for ocular administrations.

A cysteamine-zinc complex, cysteamine-magnesium complex, cysteamine-calcium complex may be formulated into a solution, emulsion or suspension dosage form for parenteral administrations.

A cysteamine-zinc complex, cysteamine-magnesium complex, cysteamine-calcium complex may be formulated into a cream, gel, lotion, stick, or ointment dosage form for topical dermal administration.

Optional pharmaceutically acceptable excipients, or excipients that have pharmaceutically acceptable safety and stability, can be included in the cysteamine-zinc complex, cysteamine-magnesium complex, cysteamine-calcium complex containing dosage forms to accommodate bioavailability, performance, stability, manufacturability, and compliance considerations.

The various embodiments of the present disclosure can be assembled in several different ways. Unless indicated otherwise, all ingredient concentrations are presented in units of % weight/volume (% w/v).

Ophthalmic compositions may be formulated such that they can be administered topically to the eye. The comfort may be maximized if it is practical, although sometimes preparation considerations (e.g. drug stability) may necessitate less than optimal comfort.

The thiol drug, such as cysteamine, may be present in any suitable amount in the pharmaceutical compositions. For oral solid and liquid compositions, the thiol drug, such as cysteamine, may be present in an amount of about 0.001-1%, about 1-2%, about 2-4%, about 4-6%, about 6-8%, about 8-10%, about 10-20%, about 20-30%, about 30-40%, about 40-50%, about 50-60%, about 60-70%, about 70-80%, about 80-90%, or higher than 90% by weight. The amounts are based upon the free drug weight without zinc.

For topical dermal liquids, creams, gels, etc., the concentration of the drug, such as cysteamine may be about 0.001-1%, about 1-2%, about 2-4%, about 4-6%, about 6-8%, about 8-10%, about 10-20%, about 20-30%, about 30-40%, about 40-50%, or higher than 50% by weight. The amounts are based upon the free drug weight without zinc.

In certain topical ophthalmic compositions, the concentration of cysteamine is from 0.1% to 2.0%, more preferably from 0.4% to 1%.

For topical ophthalmic compositions, the concentration of the drug, such as cysteamine may be about 0.001-0.1% w/v, about 0.1-0.2% w/v, about 0.2-0.3% w/v, about 0.3-0.4% w/v, about 0.4-0.5% w/v, about 0.5-0.6% w/v, about 0.6-0.7% w/v, about 0.7-0.8% w/v, about 0.8-0.9% w/v, about 0.9-1% w/v, about 1-1.1% w/v, about 1.1-1.2% w/v, about 1.2-1.3% w/v, about 1.3-1.4% w/v, about 1.4-1.5% w/v, about 1.5-1.6% w/v, about 1.6-1.7% w/v, about 1.7-1.8% w/v, about 1.8-1.9% w/v, about 1.9-2% w/v, about 0.005-0.6 molar (M), about 0.005-0.3 M, about 0.3-0.6 M, about 0.04-0.045 M, about 0.045-0.05 M, about 0.05-0.055 M, about 0.055-0.06 M, about 0.06-0.065 M, about 0.065-0.07 M, about 0.07-0.075 M, about 0.075-0.08 M, about 0.005-0.1 M, about 0.1-0.2 M, about 0.2-0.3 M, about 0.3-0.4 M, about 0.4-0.5 M, about 0.5-0.6 M, about 0.001-0.02 M, about 0.02-0.04 M, about 0.04-0.06 M, about 0.06-0.08 M, about 0.08-0.1 M, about 0.1-0.12 M, about 0.12-0.14 M, about 0.14-0.16 M, about 0.16-0.18 M, about 0.18-0.2 M, or about 0.057 M.

Zinc, a zinc compound, or a zinc salt, such as zinc chloride, for forming the complex with the drug, may be present in any suitable amount. For example, in liquid compositions, such as topical ophthalmic liquids, the zinc, zinc compound, or zinc salt, e.g. zinc chloride, may be present in a concentration of about 0.01-5% w/v, about 0.01-0.1% w/v, about 0.1-0.2% w/v, about 0.2-0.3% w/v, about 0.3-0.4% w/v, about 0.4-0.5% w/v, about 0.5-0.6% w/v, about 0.6-0.7% w/v, about 0.7-0.8% w/v, about 0.8-0.9% w/v, about 0.9-1% w/v, about 1-1.5% w/v, about 1.5-2% w/v, about 2-2.5% w/v, about 2.5-3% w/v, about 3-3.5% w/v, about 3.5-4% w/v, about 4-4.5% w/v, about 4.5-5% w/v, about 0.01-0.2% w/v, about 0.2-0.4% w/v, about 0.4-0.6% w/v, about 0.6-0.8% w/v, about 0.8-1% w/v, about 0.01-1% w/v, about 1-2% w/v, about 2-3% w/v, about 3-4% w/v, about 4-5% w/v, about 0.001-0.5 M, about 0.001-0.01 M, about 0.01-0.02 M, about 0.02-0.03 M, about 0.03-0.04 M, about 0.04-0.05 M, about 0.05-0.06 M, about 0.06-0.07 M, about 0.07-0.08 M, about 0.08-0.09 M, about 0.09-0.1 M, about 0.1-0.12 M, about 0.12-0.14 M, about 0.14-0.16 M, about 0.16-0.18 M, about 0.18-0.2 M, about 0.2-0.25 M, about 0.25-0.3 M, about 0.3-0.35 M, about 0.35-4 M, about 0.4-0.45 M, about 0.45-0.5 M, about 0.001-0.02 M, about 0.02-0.04 M, about 0.04-0.06 M, about 0.06-0.08 M, about 0.08-0.1 M, about 0.01-0.1 M, about 0.1-0.2 M, about 0.2-0.3 M, about 0.3-0.4 M, about 0.4-0.5, or about 0.03 M.

In certain embodiments, the ophthalmic composition of present disclosure has a pH in a range of about 4-7, about 4.5-6.5, about 4-5, about 5-6, about 6-7, about 4-4.5, about 4.5-5, about 5-5.5, about 5.5-6, about 6-6.5, about 6.5-7, about 4-4.2, about 4.2-4.4, about 4.4-4.6, about 4.6-4.8, about 4.8-5, about 5-5.2, about 5.2-5.4, about 5.4-5.6, about 5.6-5.8, about 5.8-6, about 6-6.2, about 6.2-6.4, about 6.4-6.6, about 6.6-6.8, or about 6.8-7.

In yet another embodiment, the ophthalmic composition of present disclosure has an osmolality from 200 to 400 milliosmoles/liter (mOsm/L), and more preferably from 240 to 360 mOsm/L.

A pharmaceutical composition according to the present disclosure may include one or more pharmaceutically acceptable buffering agents, tonicity-adjusting agents, surfactants, solubilizer, viscosity agents, pH-adjusting agents, chelating agents or combinations thereof.

A pharmaceutical composition according to the present disclosure may also contain one or more pharmaceutically acceptable preservatives.

Examples of buffering agents include, but are not limited to borate, citrate, acetate, carbonate, borate-polyol complexes, borate, acetate, e.g., sodium acetate, amino acids, salts, combinations thereof and the like. Terms such as “borate,” “citrate,” etc., refer to salt forms, as well as acid forms, base forms, or combinations thereof. For some pharmaceutical compositions, such as liquids, e.g., ophthalmic compositions, the buffering agent (e.g. citrate) may be present at a concentration of about 0.001-10% w/v, about 0.001-0.02% w/v, about 0.02-0.04% w/v, about 0.04-0.06% w/v, about 0.06-0.08% w/v, about 0.08-0.1% w/v, about 0.1-0.12% w/v, about 0.12-0.14% w/v, about 0.14-0.16% w/v, about 0.16-0.18% w/v, about 0.18-0.2% w/v, about 0.2-0.25% w/v, about 0.25-0.3% w/v, about 0.3-0.4% w/v, about 0.4-0.5% w/v, about 0.5-0.6% w/v, about 0.6-0.7% w/v, about 0.7-0.8% w/v, about 0.8-0.9% w/v, about 0.9-1% w/v, about 1-2% w/v, about 2-3% w/v, about 3-4% w/v, about 4-5% w/v, about 5-6% w/v, about 6-7% w/v, about 7-8% w/v, about 8-9% w/v, about 9-10% w/v, about 0.001-2% w/v, about 2-4% w/v, about 4-6% w/v, about 6-8% w/v, about 8-10% w/v, about 0.001-5% w/v, or about 5-10% w/v.

Examples of preservatives include, but are not limited to benzalkonium chloride, benzethonium chloride, p-oxybenzoates such as methyl p-oxybenzoate or ethyl p-oxybenzoate, benzyl alcohol, phenethyl alcohol, sorbic acid or its salt, thimerosal, chlorobutanol, other quaternary amines and the like, chlorhexidine gluconate, stabilized oxychloro complex, combinations thereof and the like. The concentration of preservative in a liquid, such as an ophthalmic liquid, may be 0.0001-1% w/v, e.g. 0.001-0.02% w/v for benzalkonium chloride or a similar preservative.

Examples of tonicity-adjusting agents include, but are not limited to mannitol, sorbitol, potassium chloride, sodium chloride, xylitol, glycerin, trehalose, taurine, erythritol, combinations thereof and the like. In some embodiments, the tonicity-adjusting agent is mannitol. In some embodiments, the tonicity-adjusting agent is sodium chloride.

Any suitable amount of tonicity-adjusting agent may be used, such as about 0.01-10% w/v, about 0.01-0.1% w/v, about 0.1-0.2% w/v, about 0.2-0.3% w/v, about 0.3-0.4% w/v, about 0.4-0.5% w/v, about 0.5-0.6% w/v, about 0.6-0.7% w/v, about 0.7-0.8% w/v, about 0.8-0.9% w/v, about 0.9-1% w/v, about 1-2% w/v, about 2-3% w/v, about 3-4% w/v, about 4-5% w/v, about 5-6% w/v, about 6-7% w/v, about 7-8% w/v, about 8-9% w/v, or about 9-10% w/v. When the tonicity-adjusting agent is mannitol, a concentration of about 0.01-4%, such as 0.01-0.5% w/v, about 0.5-1% w/v, about 1-1.5% w/v, about 1.5-2% w/v, about 2-2.5% w/v, about 2.5-3% w/v, about 3-3.5% w/v, or about 3.5-4% w/v, may be of particular interest. When the tonicity-adjusting agent is sodium chloride, a concentration of about 0.001-1% w/v, such as about 0.001-0.1% w/v, about 0.1-0.2% w/v, about 0.2-0.3% w/v, about 0.3-0.4% w/v, about 0.4-0.5% w/v, about 0.5-0.6% w/v, about 0.6-0.7% w/v, about 0.7-0.8% w/v, about 0.8-0.9% w/v, or about 0.9-1% w/v, may be of particular interest.

Examples of surfactants include, but are not limited to poloxamers, tyloxapol, polysorbate such as polysorbate 80, polysorbate 20, polyoxyethylene castor oil derivatives, sorbitan esters, combinations thereof and the like.

Examples of solubilizers include, but are not limited to solutol, soluplus, vegetable oils, or combinations thereof and the like.

Examples of viscosity agents include, but are not limited to carboxymethylcellulose, hyaluronic acid, polyvinyl alcohol, hydroxypropyl methylcellulose, polylysine, polyacrylic acid, polyacrylamides, N-(2-Hydroxypropyl) methacrylamide (HPMA), xanthan gum, pectins, chitosan, dextran, hydropropylcellulose, hydroethyl cellulose, carrageenan, guar gum, polyoxyl stearate 40, polyvinylpyrrolidone, polyethylene glycol, propylene glycol, combinations thereof and the like.

Any suitable amount of viscosity agent may be used, such as about 0.01-10%, about 0.01-0.1% w/v, about 0.1-0.2% w/v, about 0.2-0.3% w/v, about 0.3-0.4% w/v, about 0.4-0.5% w/v, about 0.5-0.6% w/v, about 0.6-0.7% w/v, about 0.7-0.8% w/v, about 0.8-0.9% w/v, about 0.9-1% w/v, about 1-2% w/v, about 2-3% w/v, about 3-4% w/v, about 4-5% w/v, about 5-6% w/v, about 6-7% w/v, about 7-8% w/v, about 8-9% w/v, or about 9-10% w/v. When the viscosity agent is a hydroxypropylmethyl cellulose, a concentration of about 0.01-1% w/v, 0.01-0.1% w/v, about 0.1-0.2% w/v, about 0.2-0.3% w/v, about 0.3-0.4% w/v, about 0.4-0.5% w/v, about 0.5-0.6% w/v, about 0.6-0.7% w/v, about 0.7-0.8% w/v, about 0.8-0.9% w/v, about 0.9-1% w/v, about 0.01-0.2% w/v, about 0.2-0.4% w/v, about 0.4-0.6% w/v, about 0.6-0.8% w/v, about 0.8-1% w/v, about 0.01-0.3% w/v, about 0.3-0.6% w/v, or about 0.6-1% w/v may be of particular interest. When the viscosity agent is a carboxymethyl cellulose, e.g. sodium carboxymethylcellulose, a concentration of about 0.5-10% w/v, 0.1-1% w/v, about 1-2% w/v, about 2-3% w/v, about 3-4% w/v, about 4-5% w/v, about 5-6% w/v, about 6-7% w/v, about 7-8% w/v, about 8-9% w/v, about 9-10% w/v, about 4-4.2% w/v, about 4.2-4.4% w/v, about 4.4-4.6% w/v, about 4.6-4.8% w/v, about 4.8-5% w/v, about 5-5.2% w/v, about 5.2-5.4% w/v, about 5.4-5.6% w/v, about 5.6-5.8% w/v, about 5.8-6% w/v, about 6-6.2% w/v, about 6.2-6.4% w/v, about 6.4-6.6% w/v, about 6.6-6.8% w/v, or about 6.8-7% w/v may be of particular interest.

Examples of the alkaline agents that may be used as pH adjusting agents, include, but are not limited to sodium hydroxide (NaOH), potassium hydroxide (KOH), tromethamine, monoethanolamine, sodium bicarbonate (NaHCO₃) and other organic and inorganic bases and the like and mixtures thereof.

Examples of the acidic agents that may be used as pH adjusting agents include but are not limited to hydrochloric acid (HCl), citric acid, tartaric acid, lactic acid, acetic acid, and other organic and inorganic acids and the like and mixtures thereof.

Examples of chelating agents include, but are not limited to sodium edetate, sodium citrate, condensed sodium phosphate, combinations thereof and the like. Any suitable amount of chelating agent, e.g. sodium edetate, may be use. For example, in liquids, such as topical ophthalmic compositions, the chelating agent, such as sodium edetate, may be present in a concentration of about 0.01-1% w/v, about 0.001-0.01% w/v, about 0.01-0.02% w/v, about 0.02-0.03% w/v, about 0.03-0.04% w/v, about 0.04-0.05% w/v, about 0.05-0.06% w/v, about 0.06-0.07% w/v, about 0.07-0.08% w/v, about 0.08-0.09% w/v, about 0.09-0.1% w/v, about 0.1-0.2% w/v, about 0.2-0.3% w/v, about 0.3-0.4% w/v, about 0.4-0.5% w/v, about 0.5-0.6% w/v, about 0.6-0.7% w/v, about 0.7-0.8% w/v, about 0.8-0.9% w/v, about 0.9-1% w/v, about 0.04-0.06% w/v, about 0.06-0.08% w/v, about 0.08-0.1% w/v, about 0.1-0.12% w/v, about 0.12-0.14% w/v, about 0.14-0.16% w/v, about 0.16-0.18% w/v, about 0.18-0.2% w/v, or about 1% w/v.

Use of the term “reference composition” is for convenience in describing some of the embodiments herein. It is used to identify a composition that is used as a reference for comparison purposes. The reference composition is identical to a composition of interest except for an identified difference. For example, in the statement “wherein the pharmaceutical composition has enhanced physicochemical or organoleptic properties of thiol drug, as compared to the thiol drug in a reference composition that it is free of zinc,” “the pharmaceutical composition” the identified difference is that the reference composition is free of zinc. Otherwise, the reference composition is identical to “the pharmaceutical composition.” For example, if the appropriate amount of zinc were added to the reference composition, it would then be “the pharmaceutical composition.”

The following embodiments are specifically contemplated:

### Embodiment 1

A pharmaceutical composition comprising: a thiol drug or a pharmaceutically acceptable salt thereof; zinc or a pharmaceutically acceptable salt thereof, magnesium or a pharmaceutically acceptable salt thereof, calcium or a pharmaceutically acceptable salt thereof, or a combination thereof; and a pharmaceutically acceptable excipient.

### Embodiment 2

The pharmaceutical composition of embodiment 1, wherein the thiol drug is cysteamine or a pharmaceutically acceptable salt thereof.

### Embodiment 3

The pharmaceutical composition of embodiment 1 or 2, wherein the pharmaceutical composition has enhanced physicochemical properties, enhanced organoleptic properties, or a combination of enhanced physicochemical properties and enhanced organoleptic properties, of the thiol drug, as compared to the thiol drug in a reference composition that is free of zinc.

### Embodiment 4

The pharmaceutical composition of embodiment 1, 2, or 3, which has improved stability of the thiol drug at 25° C. as compared to a reference composition that is free of zinc.

### Embodiment 5

The pharmaceutical composition of embodiment 1, 2, 3, or 4, comprising the pharmaceutically acceptable salt of zinc.

### Embodiment 6

The pharmaceutical composition of embodiment 5, wherein the pharmaceutically acceptable salt of zinc comprises zinc bromide, zinc chloride, zinc carbonate, zinc gluconate, zinc acetate, zinc phosphate, zinc sulfate, or a combination thereof.

### Embodiment 7

The pharmaceutical composition of embodiment 1, 2, 3, 4, 5, or 6, wherein the molar ratio of thiol drug:zinc is in a range of about 8:1 to about 1:1.

### Embodiment 8

The pharmaceutical composition of embodiment 7, wherein the molar ratio of thiol drug:zinc is in a range of about 4:1 to about 1:1.

### Embodiment 9

The pharmaceutical composition of embodiment 2, 3, 4, 5, 6, 7, or 8, which is a topical ophthalmic liquid, wherein the cysteamine or the pharmaceutically acceptable salt thereof is present at a concentration that is about 0.1% w/v to 2% w/v.

### Embodiment 10

The pharmaceutical composition of embodiment 9, wherein the cysteamine or the pharmaceutically acceptable salt thereof is present at a concentration that is about 0.4% w/v to about 1% w/v.

### Embodiment 11

The pharmaceutical composition of embodiment 1, 2, 3, 4, 5, 6, 7, or 8, which is an oral dosage form that is a tablet, a capsule, pellets, granules, a powder, or a drinkable liquid.

### Embodiment 12

The pharmaceutical composition of embodiment 11, containing about 10 mg to about 1000 mg of the cysteamine or the pharmaceutically acceptable salt thereof.

### Embodiment 13

The pharmaceutical composition of embodiment 1, 2, 3, 4, 5, 6, 7, or 8, which is a parenteral dosage form that is a solution, an emulsion, or a suspension.

### Embodiment 14

The pharmaceutical composition of embodiment 13, containing about 2% to about 20% by weight of the cysteamine or the pharmaceutically acceptable salt thereof.

### Embodiment 15

The pharmaceutical composition of embodiment 1, 2, 3, 4, 5, 6, 7, or 8, wherein the complex is in a cream, gel, lotion, stick, or ointment dosage form for topical dermal administration.

### Embodiment 16

The pharmaceutical composition of embodiment 1, 2, 3, 4, 5, 6, 7, 8, 9, 10, 11, 12, 13, 14, or 15, for the treatment of or management of cystinosis disease.

### Embodiment 17

The pharmaceutical composition of embodiment 15, for the treatment of or management of skin conditions.

### Embodiment 18

A method of treating a cystinosis disease, comprising administering the pharmaceutical composition of embodiment 1, 2, 3, 4, 5, 6, 7, 8, 9, 10, 11, 12, 13, 14, or 15 to a mammal in need thereof.

### Embodiment 19

The method of embodiment 18, wherein the mammal is a human being.

### Embodiment 20

A kit comprising the pharmaceutical composition of embodiment 1, 2, 3, 4, 5, 6, 7, 8, 9, 10, 11, 12, 13, 14, or 15 and a label, wherein the label contains instructions to administer the pharmaceutical composition to a mammal or a human being for the treatment of a cystinosis disease.

### Embodiment 21

Use of the pharmaceutical composition of embodiment 1, 2, 3, 4, 5, 6, 7, 8, 9, 10, 11, 12, 13, 14, or 15 in the manufacture of a medicament for the treatment of a cystinosis disease.

### Embodiment 22

Use of the pharmaceutical composition of embodiment 15 in the manufacture of a medicament for the treatment of skin conditions.

### Embodiment 23

A method of enhancing the stability of a thiol drug in a pharmaceutical composition, comprising combining zinc or a pharmaceutically acceptable salt thereof, magnesium or a pharmaceutically acceptable salt thereof, calcium or a pharmaceutically acceptable salt thereof, or a combination thereof, with the thiol drug, or a pharmaceutically acceptable salt thereof, and a pharmaceutically acceptable excipient.

### Embodiment 24

The method of embodiment 23, wherein the thiol drug is cysteamine or a pharmaceutically acceptable salt thereof.

### Embodiment 25

A method of improving the stability, odor, or taste of a pharmaceutical dosage form containing cysteamine, comprising including zinc or a pharmaceutically acceptable salt thereof, magnesium or a pharmaceutically acceptable salt thereof, calcium or a pharmaceutically acceptable salt thereof, or a combination thereof, in the pharmaceutical dosage form.

The following examples are provided to further elucidate the advantages and features of the present disclosure but are not intended to limit the scope of the disclosure. The examples are for illustrative purposes only. Pharmaceutical grade ingredients were used in preparing the formulations described below. The quantitative composition of these examples is presented in Table 1 below.

### Example 1

Formulations in Table 1 below were prepared by conventional methods well known in the art. The formulation panel evaluated effect of zinc on cysteamine stability in preparations prepared at various pH, different cysteamine to zinc molar ratio, different type of tonicity agent (mannitol or sodium chloride), and different type of viscosity agent (sodium carboxymethylcellulose or hydroxypropyl methylcellulose). The preparations were filled into semi-permeable polyethylene eye dropper bottles.

TABLE 1
Example Formulation Compositions

| | EY- | EY- | EY- | EY- | EY- | EY- | EY- |
|---|---|---|---|---|---|---|---|
| | 6_F16 | 6_F17 | 6_F18 | 6_F19 | 6_F20 | 6_F21 | 6_F22 |
| | Description | | | | | | |
| Ingredient | Mannitol | Mannitol | Mannitol | Mannitol | Mannitol | Mannitol | NaCMC |
| (% w/v) | pH 5.5 | pH 6.0 | pH 6.5 | pH 7.0 | CH:Zn 4:1 | CH:Zn 8:1 | w/o Zn |
| Cysteamine | 0.65 | 0.65 | 0.65 | 0.65 | 0.65 | 0.65 | 0.65 |
| HCl | | | | | | | |
| Citric Acid | 0.13 | 0.13 | 0.13 | 0.13 | 0.13 | 0.13 | 0.13 |
| Monohydrate | | | | | | | |
| Edetate | 0.1 | 0.1 | 0.1 | 0.1 | 0.1 | 0.1 | 0.1 |
| Disodium | | | | | | | |
| Zinc Chloride | 0.4 | 0.4 | 0.4 | 0.4 | 0.2 | 0.1 | 0 |
| Mannitol | 2 | 2 | 2 | 2 | 2 | 2 | 0 |
| NaCl | 0 | 0 | 0 | 0 | 0 | 0 | 0.36 |
| Benzalkonium | 0.01 | 0.01 | 0.01 | 0.01 | 0.01 | 0.01 | 0.01 |
| Chloride | | | | | | | |
| HPMC E4M | 0.5 | 0.5 | 0.5 | 0.5 | 0.5 | 0.5 | 0 |
| Na CMC 7 LPH | 0 | 0 | 0 | 0 | 0 | 0 | 5.2 |
| HCl/NaOH | 5.5 | 6 | 6.5 | 7.5 | 6 | 6 | 5 |

| | EY- | EY- | EY- | EY- | EY- | EY- | EY- |
|---|---|---|---|---|---|---|---|
| | 6_F23 | 6_F24 | 6_F25 | 6_F26 | 6_F27 | 6_F28 | 6_F29 |
| | Description | | | | | | |
| Ingredient | NaCl | NaCl | NaCl | Na Cl | NaCl | NaCl | HPMC |
| (% w/v) | pH 5.0 | pH 5.5 | pH 6.0 | pH 6.5 | CH:Zn 4:1 | CH:Zn 8:1 | w/o Zn |
| Cysteamine | 0.65 | 0.65 | 0.65 | 0.65 | 0.65 | 0.65 | 0.65 |
| HCl | | | | | | | |
| Citric Acid | 0.13 | 0.13 | 0.13 | 0.13 | 0.13 | 0.13 | 0.13 |
| Monohydrate | | | | | | | |
| Edetate | 0.1 | 0.1 | 0.1 | 0.1 | 0.1 | 0.1 | 0.1 |
| Disodium | | | | | | | |
| Zinc Chloride | 0.4 | 0.4 | 0.4 | 0.4 | 0.2 | 0.1 | 0 |
| Mannitol | 0 | 0 | 0 | 0 | 0 | 0 | 0 |
| NaCl | 0.36 | 0.36 | 0.36 | 0.36 | 0.36 | 0.36 | 0.36 |
| Benzalkonium | 0.01 | 0.01 | 0.01 | 0.01 | 0.01 | 0.01 | 0.01 |
| Chloride | | | | | | | |
| HPMC E4M | 0.5 | 0.5 | 0.5 | 0.5 | 0.5 | 0.5 | 0.5 |
| Na CMC 7 LPH | 0 | 0 | 0 | 0 | 0 | 0 | 0 |
| HCl/NaOH | 5 | 5.5 | 6 | 6.5 | 6 | 6 | 5.5 |

### Example 2

Stability studies were carried out to determine the effect of zinc on cysteamine assay and impurity level. The cysteamine assay and impurity level were measured using HPLC method. The stability study was performed at 25° C. and 40° C. storage conditions. Test formulations containing zinc chloride resulted in a lower impurity level and higher cysteamine concentration compared to those without zinc chloride.

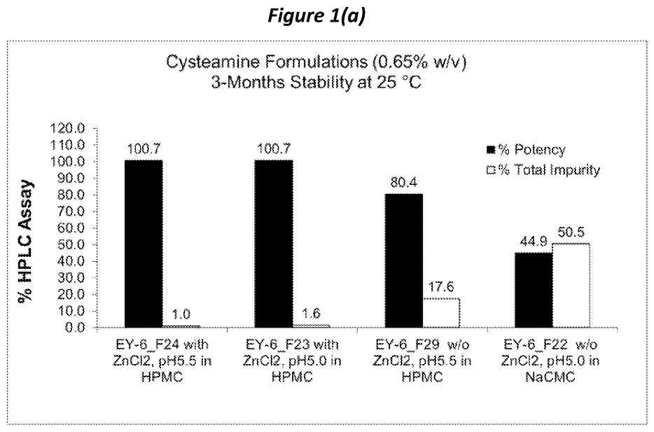

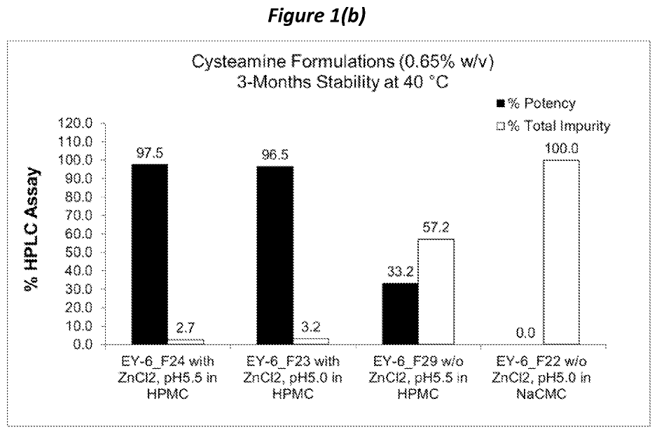

While not intending to limit the scope of the disclosure in any way, or be bound by theory, formulations with zinc chloride were significantly more stable than formulations without zinc chloride. Formulations prepared without zinc chloride were unstable regardless of viscosity agent used. Both formulations prepared without presence of zinc chloride using sodium carboxymethyl cellulose or hydroxypropyl methylcellulose were significantly less stable than those containing zinc chloride. In these formulations, sodium chloride and hydroxypropyl cellulose were used as tonicity agent and viscosity agent, respectively. The formulation pH was 5.5. The results are presented in FIG. 1(a) and FIG. 1(b).

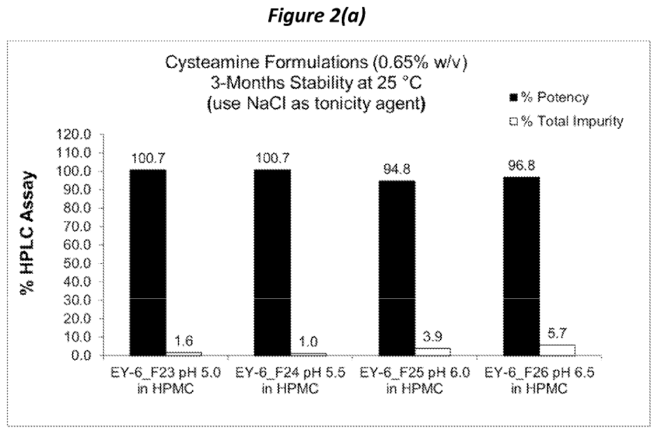

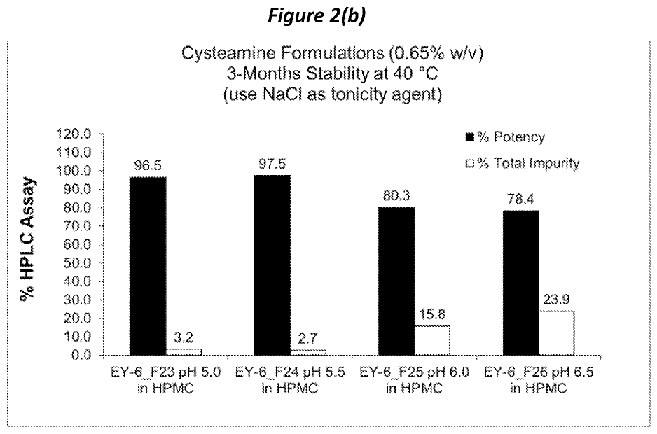

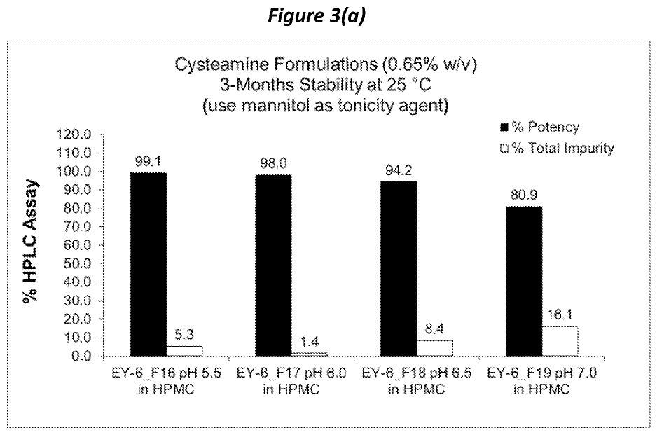

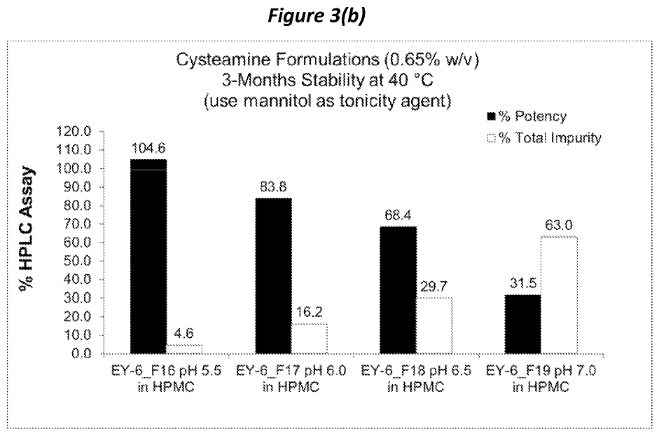

While not intending to limit the scope of the disclosure in any way, or be bound by theory, formulations prepared at lower pH were more stable than those prepared at higher pH within the studied range from pH 5 to pH 6.5. In these formulations, sodium chloride and hydroxypropyl cellulose were used as tonicity agent and viscosity agent, respectively. The results are presented in FIG. 2(a) and FIG. 2(b). A similar result was observed in formulations where mannitol and hydroxypropyl cellulose were used as tonicity agent and viscosity agent, respectively. The results are presented in FIG. 3(a) and FIG. 3(b). The studied range was pH 5.5 to pH 7.

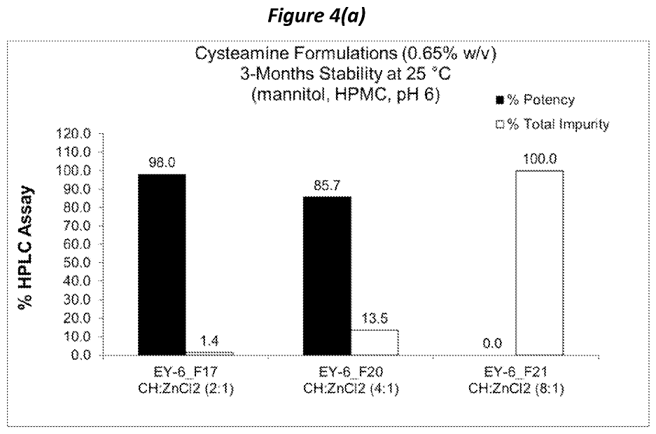

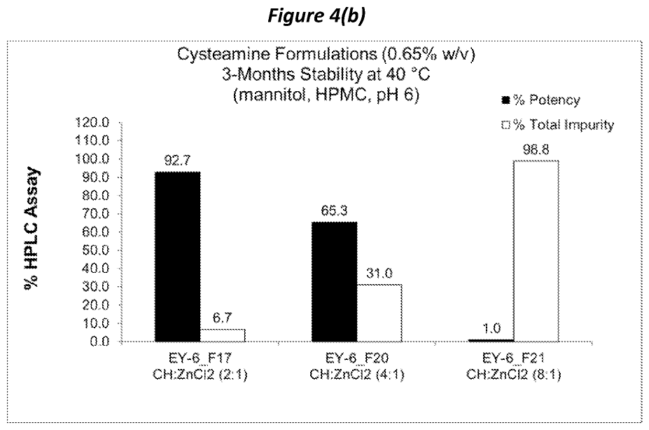

Further not intending to limit the scope of the disclosure in any way, or be bound by theory, the stabilization effect of zinc is dependent upon CH:Zn molar ratio and is in an order 2:1>4:1>8:1. The results are presented in FIG. 4(a) and FIG. 4(b).

What is claimed is:

1. A pharmaceutical composition comprising: a thiol drug or a pharmaceutically acceptable salt thereof; zinc or a pharmaceutically acceptable salt thereof, magnesium or a pharmaceutically acceptable salt thereof, calcium or a pharmaceutically acceptable salt thereof, or a combination thereof; and a pharmaceutically acceptable excipient.

2. The pharmaceutical composition of claim 1, wherein the thiol drug is cysteamine or a pharmaceutically acceptable salt thereof.

3. The pharmaceutical composition of claim 2, wherein the pharmaceutical composition has enhanced physicochemical properties, enhanced organoleptic properties, or a combination of enhanced physicochemical properties and enhanced organoleptic properties, of the thiol drug, as compared to the thiol drug in a reference composition that is free of zinc.

4. The pharmaceutical composition of claim 2, or 3, which has improved stability of the thiol drug at 25° C. as compared to a reference composition that is free of zinc.

5. The pharmaceutical composition of claim 2, 3, or 4, comprising the pharmaceutically acceptable salt of zinc.

6. The pharmaceutical composition of claim 5, wherein the pharmaceutically acceptable salt of zinc comprises zinc bromide, zinc chloride, zinc carbonate, zinc gluconate, zinc acetate, zinc phosphate, zinc sulfate, or a combination thereof.

7. The pharmaceutical composition of claim 2, 3, 4, 5, or 6, wherein the molar ratio of thiol drug:zinc is in a range of about 8:1 to about 1:1.

8. The pharmaceutical composition of claim 7, wherein the molar ratio of thiol drug:zinc is in a range of about 4:1 to about 1:1.

9. The pharmaceutical composition of claim 3, 4, 5, 6, 7, or 8, which is a topical ophthalmic liquid, wherein the cysteamine or the pharmaceutically acceptable salt thereof is present at a concentration that is about 0.1% w/v to 2% w/v.

10. The pharmaceutical composition of claim 9, wherein the cysteamine or the pharmaceutically acceptable salt thereof is present at a concentration that is about 0.4% w/v to about 1% w/v.

11. The pharmaceutical composition of claim 2, 3, 4, 5, 6, 7, or 8, which is an oral dosage form that is a tablet, a capsule, pellets, granules, a powder, or a drinkable liquid.

12. The pharmaceutical composition of claim 11, containing about 10 mg to about 1000 mg of the cysteamine or the pharmaceutically acceptable salt thereof.

13. The pharmaceutical composition of claim 2, 3, 4, 5, 6, 7, or 8, which is a parenteral dosage form that is a solution, an emulsion, or a suspension.

14. The pharmaceutical composition of claim 13, containing about 2% to about 20% by weight of the cysteamine or the pharmaceutically acceptable salt thereof.

15. The pharmaceutical composition of claim 2, 3, 4, 5, 6, 7, or 8, wherein the complex is in a cream, gel, lotion, stick, or ointment dosage form for topical dermal administration.

16. The pharmaceutical composition of claim 2, 3, 4, 5, 6, 7, 8, 9, 10, 11, 12, 13, 14, or 15, for the treatment of or management of cystinosis disease.

17. The pharmaceutical composition of claim 15, for the treatment of or management of skin conditions.

18. The pharmaceutical composition of claim 1, wherein the pharmaceutical composition has enhanced physicochemical properties, enhanced organoleptic properties, or a combination of enhanced physicochemical properties and enhanced organoleptic properties, of the thiol drug, as compared to the thiol drug in a reference composition that is free of zinc.

19. The pharmaceutical composition of claim 1, which has improved stability of the thiol drug at 25° C. as compared to a reference composition that is free of zinc.

20. The pharmaceutical composition of claim 1, comprising the pharmaceutically acceptable salt of zinc.

21. The pharmaceutical composition of claim 20, wherein the pharmaceutically acceptable salt of zinc comprises zinc bromide, zinc chloride, zinc carbonate, zinc gluconate, zinc acetate, zinc phosphate, zinc sulfate, or a combination thereof.

22. The pharmaceutical composition of claim 1, wherein the molar ratio of thiol drug:zinc is in a range of about 8:1 to about 1:1.

23. The pharmaceutical composition of claim 22, wherein the molar ratio of thiol drug:zinc is in a range of about 4:1 to about 1:1.

24. The pharmaceutical composition of claim 2, which is a topical ophthalmic liquid, wherein the cysteamine or the pharmaceutically acceptable salt thereof is present at a concentration that is about 0.1% w/v to 2% w/v.

25. The pharmaceutical composition of claim 24, wherein the cysteamine or the pharmaceutically acceptable salt thereof is present at a concentration that is about 0.4% w/v to about 1% w/v.

26. The pharmaceutical composition of claim 1, which is an oral dosage form that is a tablet, a capsule, pellets, granules, a powder, or a drinkable liquid.

27. The pharmaceutical composition of claim 26, containing about 10 mg to about 1000 mg of the cysteamine or the pharmaceutically acceptable salt thereof.

28. The pharmaceutical composition of claim 1, which is a parenteral dosage form that is a solution, an emulsion, or a suspension.

29. The pharmaceutical composition of claim 28, containing about 2% to about 20% by weight of the cysteamine or the pharmaceutically acceptable salt thereof.

30. The pharmaceutical composition of claim 1, wherein the complex is in a cream, gel, lotion, stick, or ointment dosage form for topical dermal administration.

31. The pharmaceutical composition of claim 1, the treatment of or management of cystinosis disease.

32. The pharmaceutical composition of claim 30, for the treatment of or management of skin conditions.

33. A method of treating a cystinosis disease, comprising administering the pharmaceutical composition of claim 2, 3, 4, 5, 6, 7, 8, 9, 10, 11, 12, 13, 14, 15, 16, 17, 18, 19, 20, 21, 22, 23, 24, 25, 26, 27, 28, 29, or 30 to a mammal in need thereof.

34. The method of claim 33, wherein the mammal is a human being.

35. A method of treating a cystinosis disease, comprising administering the pharmaceutical composition of claim 1, to a mammal in need thereof.

36. The method of claim 35, wherein the mammal is a human being.

37. A kit comprising the pharmaceutical composition of claim 2, 3, 4, 5, 6, 7, 8, 9, 10, 11, 12, 13, 14, 15, 16, 17, 18, 19, 20, 21, 22, 23, 24, 25, 26, 27, 28, 29, or 30, and a label, wherein the label contains instructions to administer the pharmaceutical composition to a mammal or a human being for the treatment of a cystinosis disease.

38. A kit comprising the pharmaceutical composition of claim 1, and a label, wherein the label contains instructions to administer the pharmaceutical composition to a mammal or a human being for the treatment of a cystinosis disease.

39. Use of the pharmaceutical composition of claim 2, 3, 4, 5, 6, 7, 8, 9, 10, 11, 12, 13, 14, 15, 16, 17, 18, 19, 20, 21, 22, 23, 24, 25, 26, 27, 28, 29, or 30 in the manufacture of a medicament for the treatment of a cystinosis disease.

40. Use of the pharmaceutical composition of claim 15 in the manufacture of a medicament for the treatment of skin conditions.

41. Use of the pharmaceutical composition of claim 1, in the manufacture of a medicament for the treatment of a cystinosis disease.

42. Use of the pharmaceutical composition of claim 32 in the manufacture of a medicament for the treatment of skin conditions.

43. A method of enhancing the stability of a thiol drug in a pharmaceutical composition, comprising combining zinc or a pharmaceutically acceptable salt thereof, magnesium or a pharmaceutically acceptable salt thereof, calcium or a pharmaceutically acceptable salt thereof, or a combination thereof, with the thiol drug, or a pharmaceutically acceptable salt thereof, and a pharmaceutically acceptable excipient.

44. The method of claim 43, wherein the thiol drug is cysteamine or a pharmaceutically acceptable salt thereof.

45. A method of improving the stability, odor, or taste of a pharmaceutical dosage form containing cysteamine, comprising including zinc or a pharmaceutically acceptable salt thereof, magnesium or a pharmaceutically acceptable salt thereof, calcium or a pharmaceutically acceptable salt thereof, or a combination thereof, in the pharmaceutical dosage form.
